(19) United States
(12) Patent Application Publication
(10) Pub. No.: US 2022/0370410 A1
(43) Pub. Date: Nov. 24, 2022

(54) Compositions and Methods for Treating Muscle Loss

(71) Applicant: The United States Government As Represented By The Department of Veterans Affairs, Washington, DC (US)

(72) Inventor: Jose M. Garcia, Seattle, WA (US)

(21) Appl. No.: 17/752,491

(22) Filed: May 24, 2022

Related U.S. Application Data
(60) Provisional application No. 63/192,490, filed on May 24, 2021.

(51) Int. Cl. A61K 31/405; A61P 21/00

(52) U.S. Cl. CPC A61K 31/405; A61P 21/00

(57) ABSTRACT

Disclosed are methods of treating muscle loss in a subject comprising administering a therapeutically effective amount of a GHSR1a antagonist, a GHSR1a inverse agonist, a ghrelin antagonist, or a decoy ghrelin receptor to a subject in need thereof. Disclosed are methods of inhibiting the binding of ghrelin to GHSR1a (or GHSR1a to ghrelin) in a subject comprising administering a therapeutically effective amount of GHSR1a antagonist, GHSR1a inverse agonist, ghrelin antagonist, or decoy ghrelin receptor to a subject in need thereof. Also disclosed are methods of inhibiting the binding of ghrelin to GHSR1a (or GHSR1a to ghrelin) in a subject comprising mutating GHSR1a. Disclosed are methods of lowering proteasome activity in a subject comprising administering a therapeutically effective amount of a GHSR1a antagonist, a GHSR1a inverse agonist, a ghrelin antagonist, or a decoy ghrelin receptor to a subject in need thereof. Disclosed are methods of enhancing myogenesis in a subject comprising administering a therapeutically effective amount of a GHSR1a antagonist, a GHSR1a inverse agonist, a ghrelin antagonist, or a decoy ghrelin receptor to a subject in need thereof. Disclosed are methods of increasing mitochondrial biogenesis in a subject comprising administering a therapeutically effective amount of a GHSR1a antagonist, a GHSR1a inverse agonist, a ghrelin antagonist, or a decoy ghrelin receptor to a subject in need thereof.

### CROSS-REFERENCE TO RELATED APPLICATIONS

This application claims the benefit of U.S. Provisional Patent Application No. 63/192,490, filed on May 24, 2021, each of which is incorporated by reference herein in its entirety.

### STATEMENT REGARDING FEDERALLY SPONSORED RESEARCH

This invention was made with government support under Grant Number RO3 AG040583 awarded by the National Institute on Aging. The government has certain rights in this invention.

### BACKGROUND

The number of individuals over the age of 65 will increase to approximately 80 million in the U.S. alone, representing 20% of the population by 2050. Moreover, more than 4% of the population will be >85 years of age. A hallmark of aging is a decrease in muscle function and physical performance. This often leads to increased risk of falls and fall-related injuries, loss of independence and increased frailty, disability, morbidity, length of hospital stays, healthcare costs and mortality. Sarcopenia (from Greek sarx: flesh, penia: poverty) has been defined as the “progressive loss of muscle mass and strength with a risk of adverse outcomes such as disability, poor quality of life and death”. Sarcopenia is increasingly being recognized as a key public health issue.

Starting at age 30, individuals lose 1-2% of muscle mass and function per year and by the age of 80, more than 30% is lost. The prevalence of low muscle mass and function is estimated to be between 10-25% depending on the population and method used to identify sarcopenia. In octogenarians the prevalence increases to 50%. From a clinician's perspective and a public health point of view, the loss of muscle function is much more relevant than the decrease in muscle mass since reduced muscle function is independently associated with increased risk of functional impairment, falls, disability and mortality in the elderly. The direct cost attributed to sarcopenia in the year 2000 was 1.5% of the total healthcare expenditure. It is estimated that a 10% reduction in prevalence of sarcopenia would save $1.1 billion in health-related costs. Despite its clinical relevance, no drug therapies are currently approved for the prevention or treatment of sarcopenia.

The present patent application covers the methods and effects of negatively regulating GHSR-1a by its deletion, partial agonism or antagonism on improving muscle function associated with aging and in other settings through different mechanisms.

### BRIEF SUMMARY

Disclosed are methods of treating muscle loss in a subject comprising administering a therapeutically effective amount of a GHSR1a antagonist, a GHSR1a inverse agonist, a ghrelin antagonist, or a decoy ghrelin receptor to a subject in need thereof.

Disclosed are methods of inhibiting the binding of ghrelin to GHSR1a (or GHSR1a to ghrelin) in a subject comprising administering a therapeutically effective amount of GHSR1a antagonist, GHSR1a inverse agonist, ghrelin antagonist, or decoy ghrelin receptor to a subject in need thereof. Also disclosed are methods of inhibiting the binding of ghrelin to GHSR1a (or GHSR1a to ghrelin) in a subject comprising mutating GHSR1a.

Disclosed are methods of lowering proteasome activity in a subject comprising administering a therapeutically effective amount of a GHSR1a antagonist, a GHSR1a inverse agonist, a ghrelin antagonist, or a decoy ghrelin receptor to a subject in need thereof.

Disclosed are methods of enhancing myogenesis in a subject comprising administering a therapeutically effective amount of a GHSR1a antagonist, a GHSR1a inverse agonist, a ghrelin antagonist, or a decoy ghrelin receptor to a subject in need thereof.

Disclosed are methods of increasing mitochondrial biogenesis in a subject comprising administering a therapeutically effective amount of a GHSR1a antagonist, a GHSR1a inverse agonist, a ghrelin antagonist, or a decoy ghrelin receptor to a subject in need thereof.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows the deletion of GHSR-1a leads to better treadmill performance and greater grip strength in old mice. *: significant difference vs. young with the same genotype; #: significant difference vs old with the same genotype; §: significant difference vs WT at the same age.

FIGS. 2A-2C show GHSR-1a deletion leads to; (A) increased PGC-1α, (B) lower proteasome activity, and (C) higher myogenin transcript levels in aged mice. *: different than young with the same genotype; #: different than 24m old with the same genotype; §: different than +/+ with the same age.

FIG. 3 shows a graph of body composition (g) and of food intake (g/day) in young, old, and very old mice either with or without GHSR deletion. *:different than young; # different than old; §: genotype difference.

FIG. 4 shows a graph of muscle endurance, muscle strength, and muscle mass in young, old, and very old mice either with or without GHSR deletion. *: different than young; # different than old; §: genotype difference.

FIGS. 5A and 5B show age-related muscle fiber atrophy. A) Comparison of IIA, IIB, and IIX fibers in young, old, and very old mice. *: no genotype difference B) immunohistochemistry staining for muscle fiber types (myosin heavy chain types IIA in light grey, IIB in dark grey, IIX in black).

FIG. 6 shows a graph of fiber no and a graph of fiber % in plantaris muscle.

FIG. 7 shows a graph of mRNA levels of key skeletal muscle markers of myogenesis in GHSR knockout and wt mice in young, old, and very old mice.

FIG. 8 shows oxygen consumption rate (OCR in pmol/min) as a measure of oxidative phsphorylation in GHSR knockout and wt mice in young and very old mice. *: different than young; # different than old; §: genotype difference

FIG. 9 shows a western blot and a graph depicting OXPHOS complexes (CI through V) amounts in GHSR knockout and WT mice in young and very old mice. *: different than young; # different than old; §: genotype difference.

FIG. 10 shows age-related AMPK signaling. Top graph had an N=10 and bottom graph had an N=8. *<0.05, **<0.01, ***<0.001

FIG. 11 shows age-related mitochondrial biogenesis.

FIG. 12 shows age-related mitochondrial dynamics.

FIG. 13 shows age-related mitophagy.

FIG. 14 shows age-related autophagy.

FIG. 15 shows age-related autophagy.

FIG. 16 how age affects the ubiquitin-proteasome system.

FIG. 17 shows the different in proteasome activity in GHSR WT and knock out mice.

FIG. 18 shows age-related loss of innervation.

FIG. 19 shows age-related loss of innervation occurs in both genotypes, wild type and GHSR knockout mice.

FIG. 20 shows age-related alterations in Agrin-MuSK signaling and Noggin.

FIG. 21 shows grip strength (g) vs. oxygen consumption rate (OCR).

FIG. 22 shows treadmill running time (s) vs AMPK levels and grip strength (g) vs AMPK levels.

FIG. 23 shows treadmill running time (s) vs PGC-1a or p62 and grip strength (g) vs PGC-1a or p62.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a GHSR1a antagonist” includes a plurality of such GHSR1a antagonists, reference to “the GHSR1a antagonist” is a reference to one or more GHSR1a antagonists and equivalents thereof known to those skilled in the art, and so forth.

As used herein, the term “subject” or “patient” refers to any organism to which a composition of this invention may be administered, e.g., for experimental, diagnostic, and/or therapeutic purposes. Typical subjects include animals (e.g., mammals such as non-human primates, and humans; avians; domestic household or farm animals such as cats, dogs, sheep, goats, cattle, horses and pigs; laboratory animals such as mice, rats and guinea pigs; rabbits; fish; reptiles; zoo and wild animals). Typically, “subjects” are animals, including mammals such as humans and primates; and the like.

As used herein, the term “treating” refers to partially or completely alleviating, ameliorating, relieving, preventing, delaying onset of, inhibiting or slowing progression of, reducing severity of, and/or reducing incidence of loss of muscle mass and/or function.

As used herein, “preventing” is meant to mean minimize the chance that a subject who has an increased susceptibility for developing a disease, disorder or condition will develop the disease, disorder or condition (e.g. muscle loss). For example, prevent as used herein can mean minimize the chance that a subject who has an increased susceptibility for muscle loss will lose muscle.

As used herein, the terms “administering” and “administration” refer to any method of providing a disclosed composition (e.g. a GHSR1a antagonist, a GHSR1a inverse agonist, a ghrelin antagonist, or a decoy ghrelin receptor) to a subject. Such methods are well known to those skilled in the art and include, but are not limited to: oral administration, transdermal administration, administration by inhalation, nasal administration, topical administration, intravaginal administration, ophthalmic administration, intraaural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition. In an aspect, the skilled person can determine an efficacious dose, an efficacious schedule, or an efficacious route of administration for a disclosed composition or a disclosed conjugate so as to treat a subject.

As used herein, “inverse agonist” refers to an agent that binds to the same receptor-binding site as an agonist and not only antagonizes the effects of an agonist but, moreover, exerts the opposite effect by suppressing spontaneous receptor signaling. For example, an inverse agonist binds to GHSR1a and provides an antagonistic effect.

As used herein “GHSR1a antagonist” refers to an agent binds to a receptor that will disrupt the interaction and the function of both the agonist and inverse agonist at the receptor. For example, a GHSR1a antagonist binds to GHSR1a and disrupts/prevents ghrelin or an agonist from binding to GHSR1a.

As used herein, “ghrelin antagonist” refers to an agent that binds to ghrelin and prevents ghrelin from binding to GHSR1a.

An “effective amount” of a composition is that amount of composition which is sufficient to provide a beneficial effect to the subject to which the composition is administered. The phrase “therapeutically effective amount”, as used herein, refers to an amount that is sufficient or effective to prevent or treat (delay or prevent the onset of, prevent the progression of, inhibit, decrease or reverse) a disease or condition, including alleviating symptoms of such diseases. For example, a therapeutically effective amount of a GHSR1a antagonist, a GHSR1a inverse agonist, a ghrelin antagonist, or a decoy ghrelin receptor is an amount that is sufficient to inhibit, reduce or prevent muscle loss and/or function.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Methods of Treating

Disclosed are method of treating muscle loss comprising inhibiting the of ghrelin and growth hormone secretagogue receptor 1a (GHSR1a), or inhibiting the binding of ghrelin to growth hormone secretagogue receptor 1a (GHSR1a) by administering a therapeutically effective amount of a GHSR1a antagonist, a GHSR-1a inverse agonist, a ghrelin antagonist, or a decoy ghrelin receptor to a subject in need thereof.

Disclosed are methods of treating muscle loss in a subject comprising administering a therapeutically effective amount of a GHSR1a antagonist, a GHSR1a inverse agonist, a ghrelin antagonist, or a decoy ghrelin receptor to a subject in need thereof. Muscle loss can be treated by administering an agent that binds to GHSR1a (GHSR1a antagonist, GHSR1a inverse agonist or decoy ghrelin receptor) or that binds to ghrelin (ghrelin antagonist) as long as the agent inhibits or prevents GHSR1a and ghrelin from interacting or binding.

In some aspects, muscle loss is associated with sarcopenia or cachexia.

In some aspects, the subject in need thereof is a subject that has muscle loss or is at risk for muscle loss. In some aspects, the subject in need thereof is 65 or older. Because muscle loss can be associated with age, the subject in need thereof can be 60 or older, 65 or older, 70 or older, 75 or older, 80 or older, 85 or older, 90 or older, or 95 or older. In some aspects, the subject in need thereof has or has been diagnosed with a tumor or cancer. In some aspects, the subject in need thereof has a condition associated with cachexia including, but not limited to, heart failure, liver failure, kidney failure, dementia, sepsis or other acute or chronic conditions. In some aspects, the subject in need thereof has a neurological condition.

In some aspects, the GHSR1a antagonist is a protein, nucleic acid, fatty acid, or chemical compound that binds GHSR1a and/or prevents GHSR1a activation. For example, the GHSR1a antagonist can be, but is not limited to, a liver-expressed antimicrobial peptide 2 (LEAP-2) or a LEAP-2 related compound, (d-Lys-3)-GHRP-6, JMV 2959, HM-04, or YIL 781 hydrochloride. In some aspects, the GHSR1a antagonist can be, but is not limited to, a ghrelin derivative, a ghrelin receptor inhibitor, a ghrelin receptor antibody or antigen-binding fragment thereof, or a ghrelin analog. In some aspects, a GHSR1a antagonist can be one or more of those described in WO2005114180, hereby incorporated by reference in its entirety. In some aspects, a GHSR1a antagonist can be one or more of those described in US20150297691, hereby incorporated by reference in its entirety.

In some aspects, the GHSR1a inverse agonist is a protein, nucleic acid, fatty acid, or chemical compound that binds to GHSR1a and prevents or opposes ghrelin's effects on GHSR1a. In some aspects, the GHSR1a inverse agonist can be, but is not limited to, PF 04628935, PF 05190457, MSP, or LEAP2.

In some aspects, the decoy ghrelin receptor is a receptor that binds ghrelin and does not activate GHSR1a. For example, the decoy ghrelin receptor can be a GHSR1a fusion protein. In some aspects, a GHSR1a fusion protein can be the ghrelin binding portion of GHSR1a fused to Fc thus resulting in a GHSR1a-Fc fusion protein. This allows for ghrelin binding but it does not trigger the GHSR1a pathway.

In some aspects, the ghrelin antagonist is a protein, nucleic acid, fatty acid, or chemical compound that binds to ghrelin and/or prevents ghrelin from binding to GHSR1a. In some aspects, the ghrelin antagonist can be, but is not limited to, a ghrelin antibody or antigen-binding fragment thereof, a ghrelin inhibitor, a ghrelin receptor peptide or fragment. In some aspects, a ghrelin antagonist can be one or more of those described in US20150297691, hereby incorporated by reference in its entirety.

In some aspects, the administration of the therapeutically effective amount of a GHSR1a antagonist, GHSR1a inverse agonist, ghrelin antagonist, or decoy ghrelin receptor increases a muscle function marker in the subject in need thereof. In some aspects, the muscle function marker is myogenin, myoD, Pax7, AMPK, or PGC-1α, myosin heavy chain subtype staining, atrogin, MuRF1, OCR, OXPHOS protein content, Opa1, Mfn2, Fis1, Dnm1I, p62, Bnip3, Becn1, Atg5, Map11c3b, LC3 II/I, proteasome activity, AChR, Synaptophysin, Agrn, Nog, Ncam1, Musk, treadmill performance, and grip strength.

### C. Methods of Inhibiting

Disclosed are methods of inhibiting the binding of ghrelin to GHSR1a (or GHSR1a to ghrelin) in a subject comprising administering a therapeutically effective amount of GHSR1a antagonist, GHSR1a inverse agonist, ghrelin antagonist, or decoy ghrelin receptor to a subject in need thereof. In some aspects, inhibiting or preventing GHSR1a and ghrelin from interacting or binding to each other by administering an agent that binds to GHSR1a (GHSR1a antagonist, GHSR1a inverse agonist or decoy ghrelin receptor) or that binds to ghrelin (ghrelin antagonist) can prevent activation of the ghrelin:GHSR1a pathway which is involved in muscle mass and muscle function loss.

In some aspects, the subject in need thereof is a subject that has muscle loss or is at risk for muscle loss. In some aspects, the subject in need thereof is 65 or older. Because muscle loss can be associated with age, the subject in need thereof can be 60 or older, 65 or older, 70 or older, 75 or older, 80 or older, 85 or older, 90 or older, or 95 or older. In some aspects, the subject in need thereof has or has been diagnosed with a tumor or cancer. In some aspects, the subject in need thereof has a condition associated with cachexia including, but not limited to, heart failure, liver failure, kidney failure, dementia, sepsis or other acute or chronic conditions. In some aspects, the subject in need thereof has a neurological condition.

In some aspects, the GHSR1a antagonist is a protein, nucleic acid, fatty acid, or chemical compound that binds GHSR1a and/or prevents GHSR1a activation. For example, the GHSR1a antagonist can be, but is not limited to, a liver-expressed antimicrobial peptide 2 (LEAP-2) or a LEAP-2 related compound, (d-Lys-3)-GHRP-6, JMV 2959, HM-04, or YIL 781 hydrochloride. In some aspects, the GHSR1a antagonist can be, but is not limited to, a ghrelin derivative, a ghrelin receptor inhibitor, a ghrelin receptor antibody or antigen-binding fragment thereof, or a ghrelin analog. In some aspects, a GHSR1a antagonist can be one or more of those described in WO2005114180, hereby incorporated by reference in its entirety. In some aspects, a GHSR1a antagonist can be one or more of those described in US20150297691, hereby incorporated by reference in its entirety.

In some aspects, the GHSR1a inverse agonist is a protein, nucleic acid, fatty acid, or chemical compound that binds to GHSR1a and prevents or opposes ghrelin's effects on GHSR1a. In some aspects, the GHSR1a inverse agonist can be, but is not limited to, PF 04628935, PF 05190457, MSP, or LEAP2.

In some aspects, the decoy ghrelin receptor is a receptor that binds ghrelin and does not activate GHSR1a. For example, the decoy ghrelin receptor can be a GHSR1a fusion protein. In some aspects, a GHSR1a fusion protein can be the ghrelin binding portion of GHSR1a fused to Fc thus resulting in a GHSR1a-Fc fusion protein. This allows for ghrelin binding but it does not trigger the GHSR1a pathway.

In some aspects, the ghrelin antagonist is a protein, nucleic acid, fatty acid, or chemical compound that binds to ghrelin and/or prevents ghrelin from binding to GHSR1a. In some aspects, the ghrelin antagonist can be, but is not limited to, a ghrelin antibody or antigen-binding fragment thereof, a ghrelin inhibitor, a ghrelin receptor peptide or fragment. In some aspects, a ghrelin antagonist can be one or more of those described in US20150297691, hereby incorporated by reference in its entirety.

Also disclosed are methods of inhibiting the binding of ghrelin to GHSR1a or GHSR1a to ghrelin in a subject comprising mutating GHSR1a. In some aspects, a mutated GHSR1a is an altered (i.e. non-wild type) GHSR1a that due to the alteration can no longer interact with or bind to ghrelin.

In some aspects, the mutating occurs in vitro or in vivo.

In some aspects, mutating GHSR1a comprises introducing a deletion or substitution in the amino acid sequence of GHSR1a resulting in anon-functional GHSR1a. In some aspects, mutating GHSR1a comprises introducing a deletion or substitution in the nucleic acid sequence of GHSR1a which results in a mutated amino acid sequence. In some aspects, a deletion in the amino acid sequence or nucleic acid sequence is a full deletion of GHSR1a (i.e. the entire gene/protein is deleted). In some aspects, a deletion in the amino acid sequence or nucleic acid sequence is a partial deletion of GHSR1a, wherein the partial deletion results in a non-functional GHSR1a. In some aspects, a partial deletion comprises deleting 1, 2, 3, 4, 5, 6, 7, 8, 9, 10, 12, 14, 16, 18, 20, 25, 30, 35, 40, 45, 50, or more nucleic acids from the wild type gene sequence. In some aspects, a partial deletion comprises deleting 1, 2, 3, 4, 5, 6, 7, 8, 9, 10, 12, 14, 16, 18, 20, 25, 30, 35, 40, 45, 50, or more amino acids from the wild type protein. In some aspects, mutating GHSR1a comprises inserting a mutation in the GHSR1a gene using CRISPR.

### D. Methods of Lowering Proteasome Activity

Disclosed are methods of lowering proteasome activity in a subject comprising administering a therapeutically effective amount of a GHSR1a antagonist, a GHSR1a inverse agonist, a ghrelin antagonist, or a decoy ghrelin receptor to a subject in need thereof. In some aspects, lowering proteasome activity, which is a pathway for protein degradation, prevents or reduces muscle loss by maintaining protein levels required for muscle mass and function. Thus, in some aspects, protein degradation in skeletal muscle of the subject is decreased.

In some aspects, the subject in need thereof is a subject that has muscle loss or is at risk for muscle loss. In some aspects, the subject in need thereof is 65 or older. Because muscle loss can be associated with age, the subject in need thereof can be 60 or older, 65 or older, 70 or older, 75 or older, 80 or older, 85 or older, 90 or older, or 95 or older. In some aspects, the subject in need thereof has or has been diagnosed with a tumor or cancer. In some aspects, the subject in need thereof has a condition associated with cachexia including, but not limited to, heart failure, liver failure, kidney failure, dementia, sepsis or other acute or chronic conditions. In some aspects, the subject in need thereof has a neurological condition.

In some aspects, the GHSR1a antagonist is a protein, nucleic acid, fatty acid, or chemical compound that binds GHSR1a and/or prevents GHSR1a activation. For example, the GHSR1a antagonist can be, but is not limited to, a liver-expressed antimicrobial peptide 2 (LEAP-2) or a LEAP-2 related compound, (d-Lys-3)-GHRP-6, JMV 2959, HM-04, or YIL 781 hydrochloride. In some aspects, the GHSR1a antagonist can be, but is not limited to, a ghrelin derivative, a ghrelin receptor inhibitor, a ghrelin receptor antibody or antigen-binding fragment thereof, or a ghrelin analog. In some aspects, a GHSR1a antagonist can be one or more of those described in WO2005114180, hereby incorporated by reference in its entirety. In some aspects, a GHSR1a antagonist can be one or more of those described in US20150297691, hereby incorporated by reference in its entirety.

In some aspects, the GHSR1a inverse agonist is a protein, nucleic acid, fatty acid, or chemical compound that binds to GHSR1a and prevents or opposes ghrelin's effects on GHSR1a. In some aspects, the GHSR1a inverse agonist can be, but is not limited to, PF 04628935, PF 05190457, MSP, or LEAP2.

In some aspects, the decoy ghrelin receptor is a receptor that binds ghrelin and does not activate GHSR1a. For example, the decoy ghrelin receptor can be a GHSR1a fusion protein. In some aspects, a GHSR1a fusion protein can be the ghrelin binding portion of GHSR1a fused to Fc thus resulting in a GHSR1a-Fc fusion protein. This allows for ghrelin binding but it does not trigger the GHSR1a pathway.

In some aspects, the ghrelin antagonist is a protein, nucleic acid, fatty acid, or chemical compound that binds to ghrelin and/or prevents ghrelin from binding to GHSR1a. In some aspects, the ghrelin antagonist can be, but is not limited to, a ghrelin antibody or antigen-binding fragment thereof, a ghrelin inhibitor, a ghrelin receptor peptide or fragment. In some aspects, a ghrelin antagonist can be one or more of those described in US20150297691, hereby incorporated by reference in its entirety.

### E. Methods of Enhancing Myogenesis

Disclosed are methods of enhancing myogenesis in a subject comprising administering a therapeutically effective amount of a GHSR1a antagonist, a GHSR1a inverse agonist, a ghrelin antagonist, or a decoy ghrelin receptor to a subject in need thereof. In some aspects, the methods for enhancing the formation of muscle tissue can be used as a treatment for subjects having muscle loss or at risk for having muscle loss.

In some aspects, the subject in need thereof is a subject that has muscle loss or is at risk for muscle loss. In some aspects, the subject in need thereof is 65 or older. Because muscle loss can be associated with age, the subject in need thereof can be 60 or older, 65 or older, 70 or older, 75 or older, 80 or older, 85 or older, 90 or older, or 95 or older. In some aspects, the subject in need thereof has or has been diagnosed with a tumor or cancer. In some aspects, the subject in need thereof has a condition associated with cachexia including, but not limited to, heart failure, liver failure, kidney failure, dementia, sepsis or other acute or chronic conditions. In some aspects, the subject in need thereof has a neurological condition.

In some aspects, the GHSR1a antagonist is a protein, nucleic acid, fatty acid, or chemical compound that binds GHSR1a and/or prevents GHSR1a activation. For example, the GHSR1a antagonist can be, but is not limited to, a liver-expressed antimicrobial peptide 2 (LEAP-2) or a LEAP-2 related compound, (d-Lys-3)-GHRP-6, JMV 2959, HM-04, or YIL 781 hydrochloride. In some aspects, the GHSR1a antagonist can be, but is not limited to, a ghrelin derivative, a ghrelin receptor inhibitor, a ghrelin receptor antibody or antigen-binding fragment thereof, or a ghrelin analog. In some aspects, a GHSR1a antagonist can be one or more of those described in WO2005114180, hereby incorporated by reference in its entirety. In some aspects, a GHSR1a antagonist can be one or more of those described in US20150297691, hereby incorporated by reference in its entirety.

In some aspects, the GHSR1a inverse agonist is a protein, nucleic acid, fatty acid, or chemical compound that binds to GHSR1a and prevents or opposes ghrelin's effects on GHSR1a. In some aspects, the GHSR1a inverse agonist can be, but is not limited to, PF 04628935, PF 05190457, MSP, or LEAP2.

In some aspects, the decoy ghrelin receptor is a receptor that binds ghrelin and does not activate GHSR1a. For example, the decoy ghrelin receptor can be a GHSR1a fusion protein. In some aspects, a GHSR1a fusion protein can be the ghrelin binding portion of GHSR1a fused to Fc thus resulting in a GHSR1a-Fc fusion protein. This allows for ghrelin binding but it does not trigger the GHSR1a pathway.

In some aspects, the ghrelin antagonist is a protein, nucleic acid, fatty acid, or chemical compound that binds to ghrelin and/or prevents ghrelin from binding to GHSR1a. In some aspects, the ghrelin antagonist can be, but is not limited to, a ghrelin antibody or antigen-binding fragment thereof, a ghrelin inhibitor, a ghrelin receptor peptide or fragment. In some aspects, a ghrelin antagonist can be one or more of those described in US20150297691, hereby incorporated by reference in its entirety.

### F. Methods of Increasing Mitochondrial Biogenesis

Disclosed are methods of increasing mitochondrial biogenesis in a subject comprising administering a therapeutically effective amount of a GHSR1a antagonist, a GHSR1a inverse agonist, a ghrelin antagonist, or a decoy ghrelin receptor to a subject in need thereof. In some aspects, increasing mitochondrial biogenesis leads to increased mitochondria which convert energy into ATP wherein the energy can be used to build muscles. Thus, the methods for increasing mitochondrial biogenesis can be used as a treatment for subjects having muscle loss or at risk for having muscle loss.

In some aspects, the subject in need thereof is a subject that has muscle loss or is at risk for muscle loss. In some aspects, the subject in need thereof is 65 or older. Because muscle loss can be associated with age, the subject in need thereof can be 60 or older, 65 or older, 70 or older, 75 or older, 80 or older, 85 or older, 90 or older, or 95 or older. In some aspects, the subject in need thereof has or has been diagnosed with a tumor or cancer. In some aspects, the subject in need thereof has a condition associated with cachexia including, but not limited to, heart failure, liver failure, kidney failure, dementia, sepsis or other acute or chronic conditions. In some aspects, the subject in need thereof has a neurological condition.

In some aspects, the GHSR1a antagonist is a protein, nucleic acid, fatty acid, or chemical compound that binds GHSR1a and/or prevents GHSR1a activation. For example, the GHSR1a antagonist can be, but is not limited to, a liver-expressed antimicrobial peptide 2 (LEAP-2) or a LEAP-2 related compound, (d-Lys-3)-GHRP-6, JMV 2959, HM-04, or YIL 781 hydrochloride. In some aspects, the GHSR1a antagonist can be, but is not limited to, a ghrelin derivative, a ghrelin receptor inhibitor, a ghrelin receptor antibody or antigen-binding fragment thereof, or a ghrelin analog. In some aspects, a GHSR1a antagonist can be one or more of those described in WO2005114180, hereby incorporated by reference in its entirety. In some aspects, a GHSR1a antagonist can be one or more of those described in US20150297691, hereby incorporated by reference in its entirety.

In some aspects, the GHSR1a inverse agonist is a protein, nucleic acid, fatty acid, or chemical compound that binds to GHSR1a and prevents or opposes ghrelin's effects on GHSR1a. In some aspects, the GHSR1a inverse agonist can be, but is not limited to, PF 04628935, PF 05190457, MSP, or LEAP2.

In some aspects, the decoy ghrelin receptor is a receptor that binds ghrelin and does not activate GHSR1a. For example, the decoy ghrelin receptor can be a GHSR1a fusion protein. In some aspects, a GHSR1a fusion protein can be the ghrelin binding portion of GHSR1a fused to Fc thus resulting in a GHSR1a-Fc fusion protein. This allows for ghrelin binding but it does not trigger the GHSR1a pathway.

In some aspects, the ghrelin antagonist is a protein, nucleic acid, fatty acid, or chemical compound that binds to ghrelin and/or prevents ghrelin from binding to GHSR1a. In some aspects, the ghrelin antagonist can be, but is not limited to, a ghrelin antibody or antigen-binding fragment thereof, a ghrelin inhibitor, a ghrelin receptor peptide or fragment. In some aspects, a ghrelin antagonist can be one or more of those described in US20150297691, hereby incorporated by reference in its entirety.

### G. Compositions and Administration

Disclosed are compositions comprising a GHSR1a antagonist, a GHSR1a inverse agonist, a ghrelin antagonist, or a decoy ghrelin receptor or a pharmaceutically acceptable salt thereof.

1. Pharmaceutical Compositions

In some aspects, the disclosed compositions can be pharmaceutical compositions. Disclosed are compositions comprising a GHSR1a antagonist, a GHSR1a inverse agonist, a ghrelin antagonist, or a decoy ghrelin receptor in combination with a pharmaceutically acceptable carrier. For example, in some aspects, disclosed are pharmaceutical compositions comprising a composition comprising a GHSR1a antagonist, a GHSR1a inverse agonist, a ghrelin antagonist, or a decoy ghrelin receptor and a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidylcholine (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, Pa. 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, or conjugate of the invention is not compromised. Pharmaceutical compositions may also include one or more active ingredients (in addition to the composition of the invention) such as antimicrobial agents, anti-inflammatory agents, anesthetics, and the like.

The pharmaceutical compositions as disclosed herein can be prepared for oral or parenteral administration. Pharmaceutical compositions prepared for parenteral administration include those prepared for intravenous (or intra-arterial), intramuscular, subcutaneous, intraperitoneal, transmucosal (e.g., intranasal, intravaginal, or rectal), or transdermal (e.g., topical) administration. Aerosol inhalation can also be used to deliver the fusion proteins. Thus, compositions can be prepared for parenteral administration that includes fusion proteins dissolved or suspended in an acceptable carrier, including but not limited to an aqueous carrier, such as water, buffered water, saline, buffered saline (e.g., PBS), and the like. One or more of the excipients included can help approximate physiological conditions, such as pH adjusting and buffering agents, tonicity adjusting agents, wetting agents, detergents, and the like. Where the compositions include a solid component (as they may for oral administration), one or more of the excipients can act as a binder or filler (e.g., for the formulation of a tablet, a capsule, and the like). Where the compositions are formulated for application to the skin or to a mucosal surface, one or more of the excipients can be a solvent or emulsifier for the formulation of a cream, an ointment, and the like.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

The pharmaceutical compositions can be sterile and sterilized by conventional sterilization techniques or sterile filtered. Aqueous solutions can be packaged for use as is, or lyophilized, the lyophilized preparation, which is encompassed by the present disclosure, can be combined with a sterile aqueous carrier prior to administration. The pH of the pharmaceutical compositions typically will be between 3 and 11 (e.g., between about 5 and 9) or between 6 and 8 (e.g., between about 7 and 8). The resulting compositions in solid form can be packaged in multiple single dose units, each containing a fixed amount of the above-mentioned agent or agents, such as in a sealed package of tablets or capsules. The composition in solid form can also be packaged in a container for a flexible quantity, such as in a squeezable tube designed for a topically applicable cream or ointment.

The pharmaceutical compositions described above can be formulated to include a therapeutically effective amount of a composition disclosed herein. In some aspects, therapeutic administration encompasses prophylactic applications. Based on genetic testing and other prognostic methods, a physician in consultation with their patient can choose a prophylactic administration where the patient has a clinically determined predisposition or increased susceptibility (in some cases, a greatly increased susceptibility) to muscle loss.

The pharmaceutical compositions described herein can be administered to the subject (e.g., a human subject or human patient) in an amount sufficient to delay, reduce, or preferably prevent the onset of muscle loss. Accordingly, in some aspects, the subject is a human subject. In therapeutic applications, compositions are administered to a subject (e.g., a human subject) already with or diagnosed with muscle loss in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a pharmaceutical composition can be an amount that achieves a cure, but that outcome is only one among several that can be achieved. As noted, a therapeutically effective amount includes amounts that provide a treatment in which the onset or progression of the muscle loss is delayed, hindered, or prevented, or the muscle loss is ameliorated. One or more of the symptoms can be less severe. Recovery can be accelerated in an individual who has been treated.

The total effective amount of the a GHSR1a antagonist, a GHSR1a inverse agonist, a ghrelin antagonist, or a decoy ghrelin receptor in the pharmaceutical compositions disclosed herein can be administered to a mammal as a single dose, either as a bolus or by infusion over a relatively short period of time, or can be administered using a fractionated treatment protocol in which multiple doses are administered over a more prolonged period of time (e.g., a dose every 4-6, 8-12, 14-16, or 18-24 hours, or every 2-4 days, 1-2 weeks, or once a month). Alternatively, continuous intravenous infusions sufficient to maintain therapeutically effective concentrations in the blood are also within the scope of the present disclosure.

The pharmaceutical composition may be administered in a number of ways depending on whether local or systemic treatment is desired, and on the area to be treated.

### EXAMPLES

### A. Example 1

1. Background

To date, there are no FDA-approved treatments for sarcopenia. Hence, therapies for this condition are desperately needed. Other efforts in targeting different pathways are underway including androgen receptor modulators (EP2289503A3, U.S. Pat. No. 7,772,433B2), tacrines (U.S. Ser. No. 10/668,087B2) and ghrelin agonists (U.S. Pat. No. 8,710,089B2). Ghrelin agonists are thought to increase muscle mass. Surprisingly, it has been shown that ghrelin deletion prevented muscle function loss in old mice (PMID: 28585250). However, the mechanisms mediating these effects are not fully understood. Data indicates that not all effects of ghrelin are mediated through its only known receptor: GHSR-1a. This is very relevant because ghrelin, GHSR-1a agonists and antagonists are in clinical development. Described herein are methods and effects of negatively regulating GHSR-1a by its deletion, partial agonism or antagonism on improving muscle function associated with aging and in other settings through different mechanisms.

2. Description

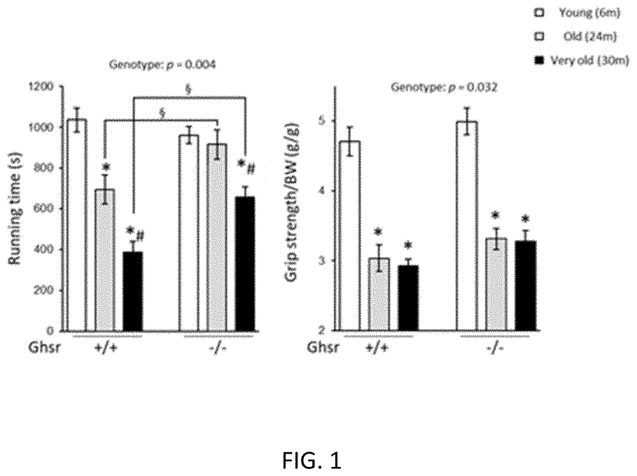

The present disclosure describes targeting the GHSR-1a by deletion, antagonism, inverse agonism, decoy receptors or other methods to prevent muscle function loss in the setting of aging, cachexia, neurologic diseases among other conditions. The data shows that deletion of GHSR-1a prevents muscle function loss seen in old mice (FIG. 1).

GHSR-1a wild-type (+/+) and knockout (−/−) mice at 5 months (white), 24 months (grey) and 30 months of age (black) were evaluated for treadmill performance and grip strength. Although there was no difference between genotypes at 5 months of age, 24 and 30 month-old GHSR-1a−/− mice had better treadmill performance than GHSR-1a+/+ mice of the same ages. Also, grip strength was higher in KO compared to WT mice at all ages.

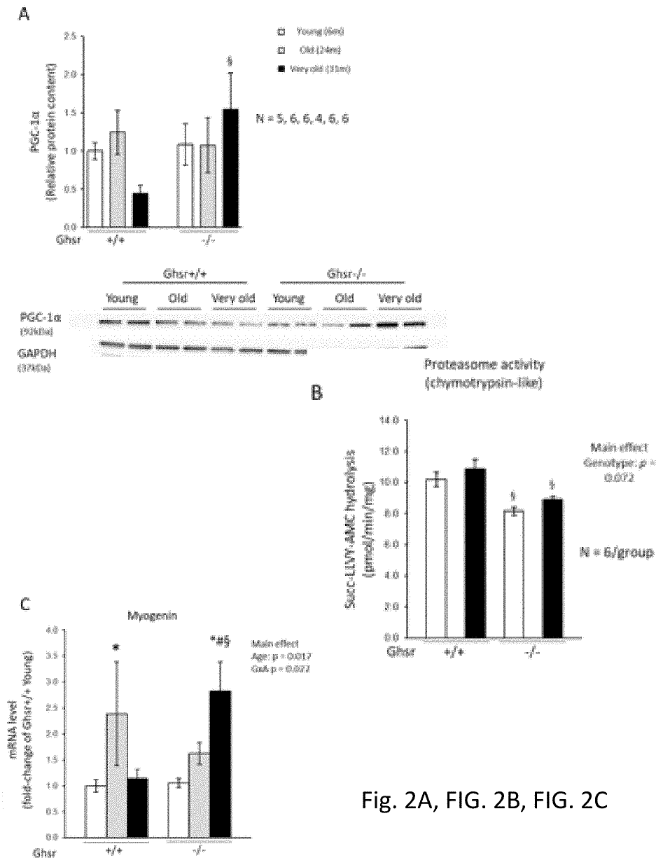

The molecular mechanisms underlying these effects has also been investigated. GHSR-1a deletion leads to significantly improved mitochondrial biogenesis, lower proteasome activity, the main pathway for protein degradation in skeletal muscle, and enhanced myogenesis as shown by myogenin and other transcript levels (FIG. 2A-C).

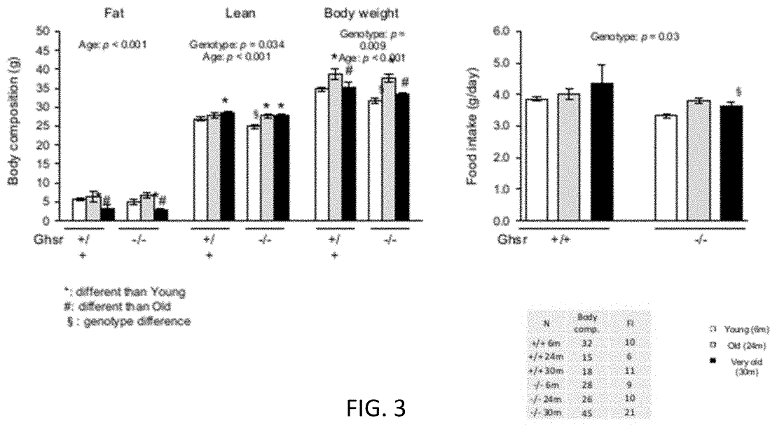

FIG. 3 shows a lower lean body mass and body weight in young mice with GHSR deletion and less food intake in aged mice with GHSR deletion.

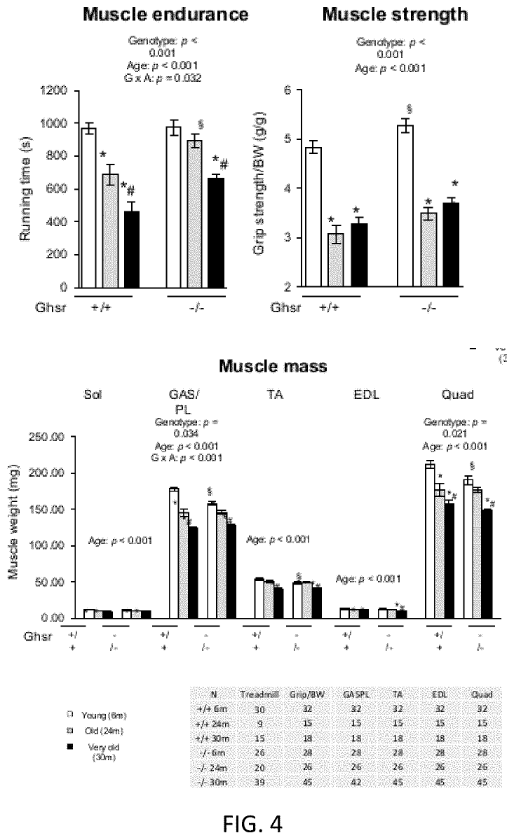

FIG. 4 shows GHSR deletion attenuates age-related declines in endurance and muscle strength, but not muscle mass.

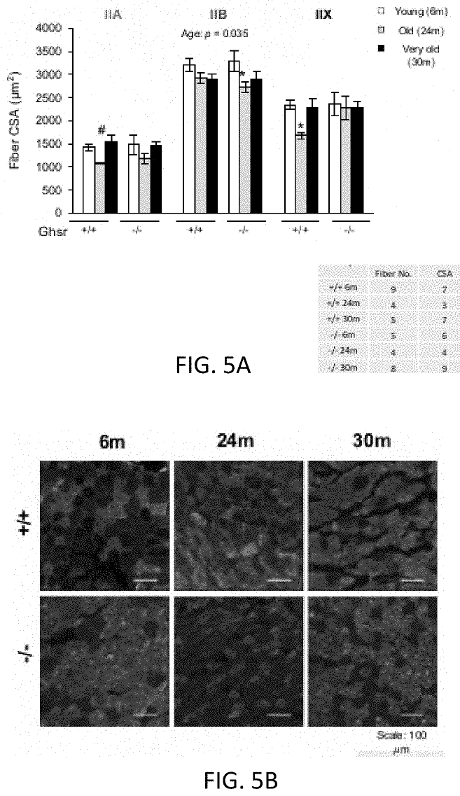

FIG. 5 shows age-related muscle fiber atrophy is more prominent in fast-twitch fibers. At 24m IIB fibers have more significant decrease in GHSR knock out mice and IIX fibers have more significant decrease in wild type.

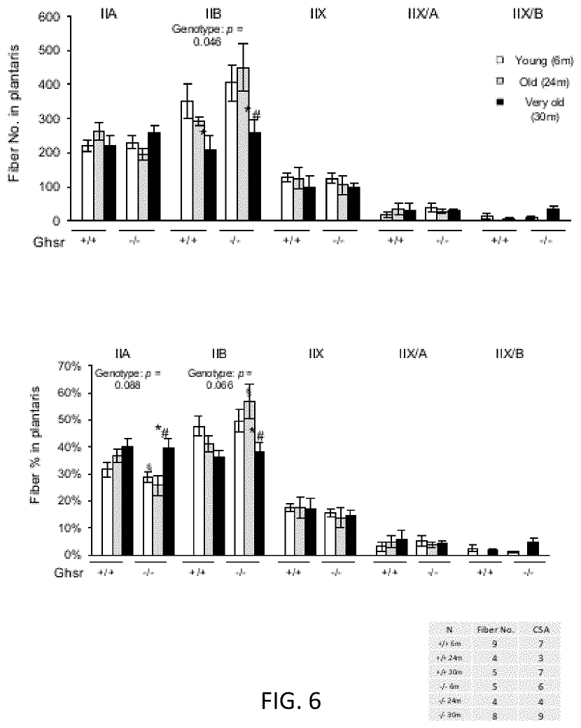

FIG. 6 shows GHSR deletion attenuates age-related decline sin muscle fiber number at 24m. GHSR knock out mice showed more IIB fibers than WT mice in plantaris muscle.

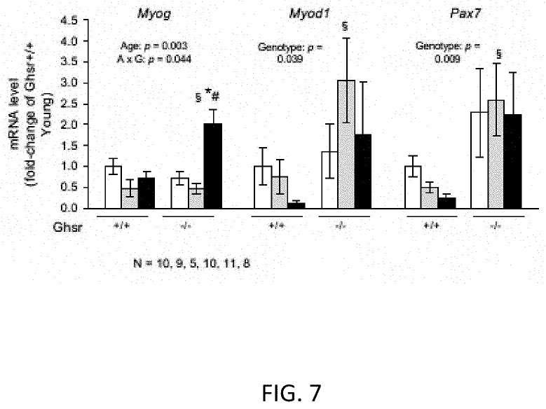

FIG. 7 looks at myogenesis markers. In, GHSR knockout mice, Myog, Myod1 and Pax7 mRNA levels were increased compared to WT mice indicating an increase in myogenesis that mediate the improved muscle function seen in these GHSR-1a KO mice.

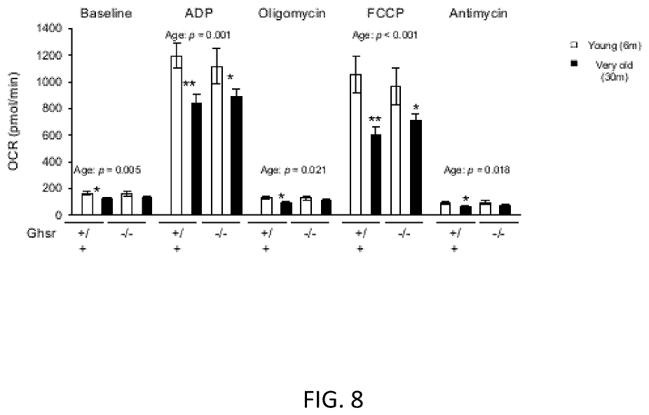

FIG. 8 shows age-related impairment in mitochondrial respiration was more prominent in GHSR WT mice. Mitochodria were isolated from plantaris muscle. No genotype difference was seen but there was a difference between young and very old mice.

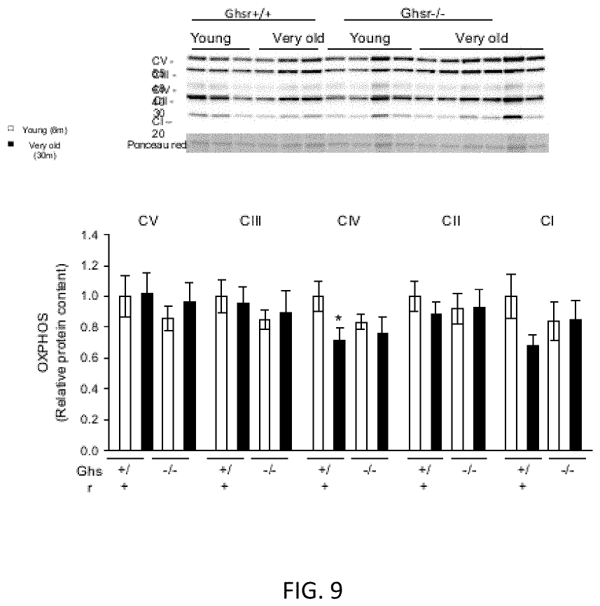

FIG. 9 shows age-related OXPHOS decrease was only found in GSHR+/+ mice.

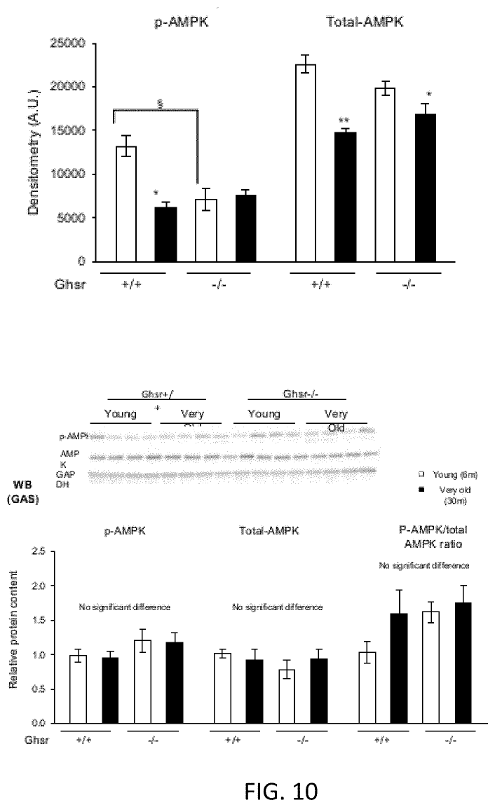

FIG. 10 shows age-related AMPK signaling. Young GHSR deficient mice show lower p-AMPK levels than wild type mice. An age-related decrease in p-AMPK (MSD assay) was only seen in WT mice. Different cohorts and muscles were used for MSD assay and western blot.

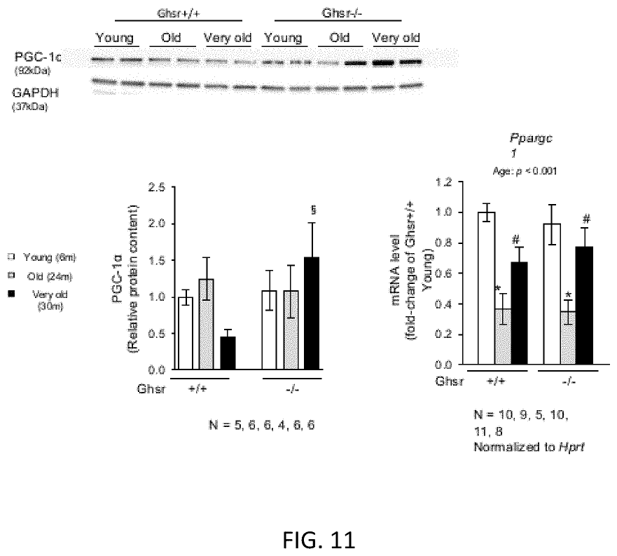

FIG. 11 shows age-related mitochondrial biogenesis. Decreased PGC-1a (transcript levels) was seen at 24m compared to young mice. Higher PGC-1a (protein levels) content was seen in very old GHSR knock out mice. This data indicates post-translational regulation.

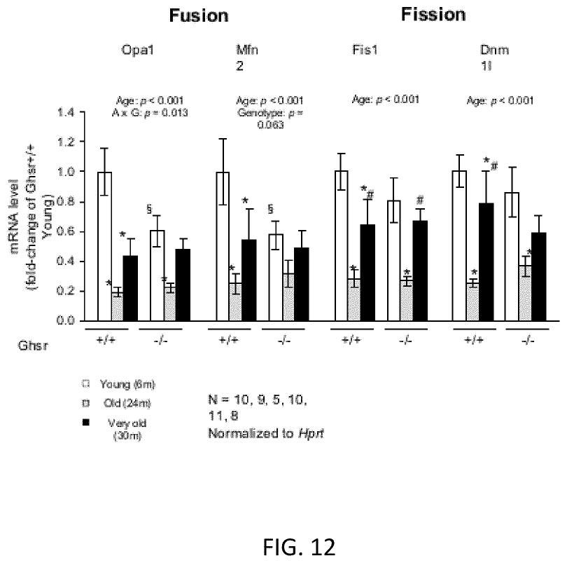

FIG. 12 shows age-related mitochondrial dynamics (fusion and fission). Lower levels of fusion genes were seen in young GHSR knock out mice. Fission and fusion genes were decreased with aging as seen in the old (and very old) mice compared to young mice.

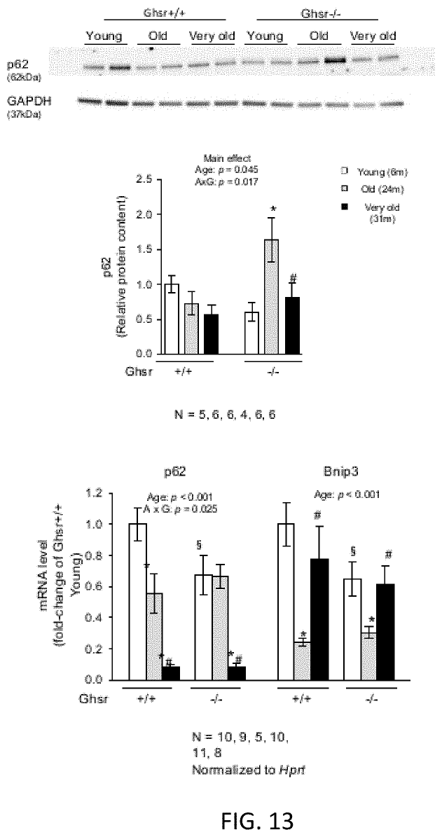

FIG. 13 shows age-related mitophagy. Lower mitophagy levels (protein and gene levels) were seen in young GHSR knock out mice. p62 (protein levels) decreases with aging in wild type mice and increases with aging in 24m GHSR knock out mice. Protein and transcript levels were not consistent in these experiments, thus indicating post-trancriptional regulation.

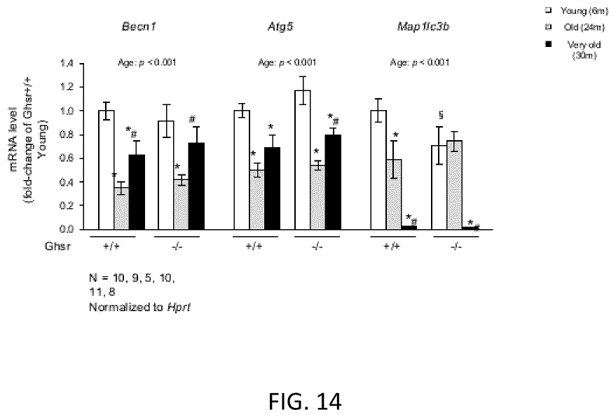

FIG. 14 shows age-related autophagy. Becn1 and Atg5 show an age-related decrease in mRNA levels. The decrease was more prominent in old mice but was also present in very old mice.

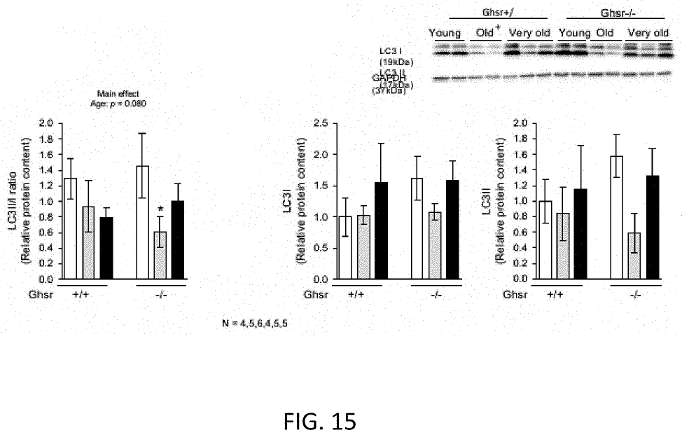

FIG. 15 also shows age-related autophagy. There is a lower LC3II/I ratio with aging. There is no genotype difference in the lower LC3II/I ratio with aging.

This data shows that GHSR deletion attenuates age-related decline in muscle function. The age-related decrease in mitochondrial biogenesis and mitophagy is attenuated by GHSR deletion indicating that GHSR plays a role in age-related mitochondrial modification.

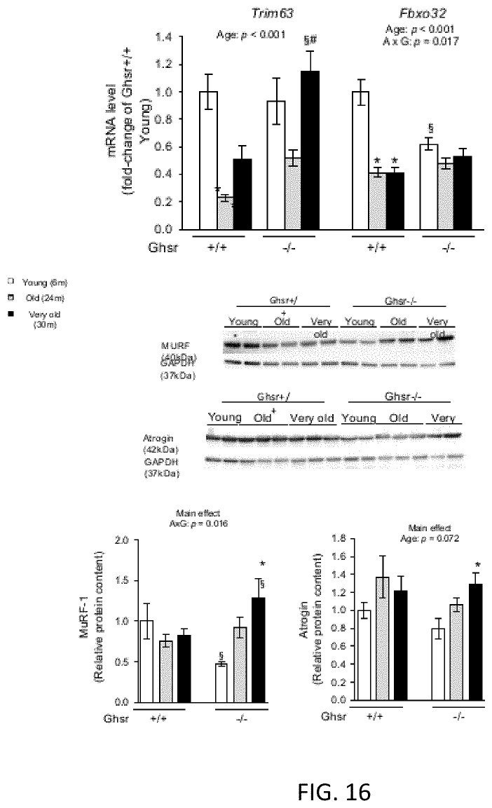

The ubiquitin-proteasome system also plays a role in age-related muscle loss. As seen in FIG. 16, the ubiquitin-proteasome system increased with aging in GHSR knock out mice. There was a lower level of MuRF-1 in young GHSR knock out mice.

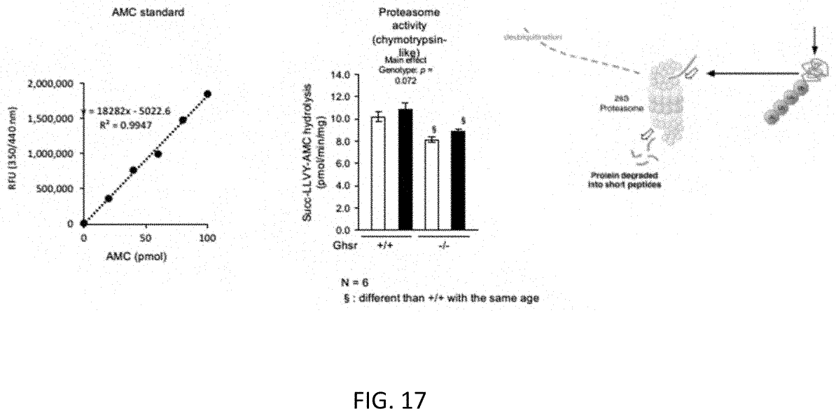

FIG. 17 shows that proteasome activity is altered in GHSR knockout mice compared to WT mice. Age did not play a role in proteasome activity but the presence or absence of GHSR did.

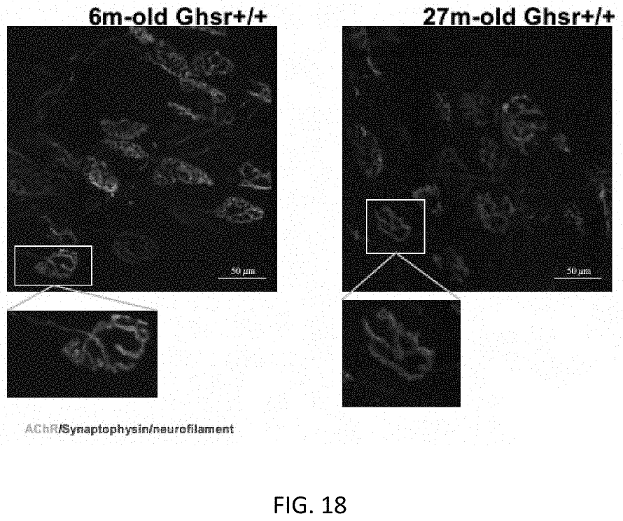

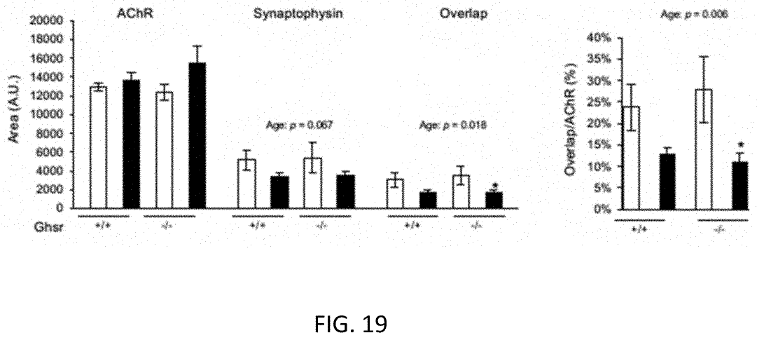

FIG. 18 shows age-related loss of innervation. The histology staining shows a loss of nerve related markers in the 27m WT mice compared to the 6m WT mice. FIG. 19 shows age-related loss of innervation occurs in both genotypes, wild type and GHSR knockout mice. Regardless of the presence or absence of GHSR, loss of nerve markers were seen in all older mice compared to younger mice.

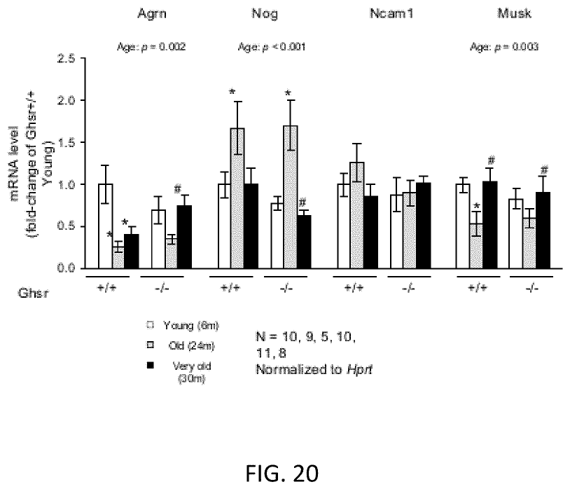

FIG. 20 shows age-related alterations in Agrin-MuSK signaling and Noggin. Agrin-LRP-MuSK signaling pathway is essential for neuromuscular junction (NMJ) formation and maintenance. Noggin is an inflammation-related BMP inhibitor which blocks the actions of BMPs on muscle fiber and motor neurons, thus contributing to disruption of NMJ. Ncam1 is a postsynaptic molecule. It is absent in adult myofibers and activated by denervation. FIG. 20 shows an age-related decrease occurs in Agrn and Musk in WT 24m mice. Noggin increases in both the GHSR+/+ and −/− mice at 24m.

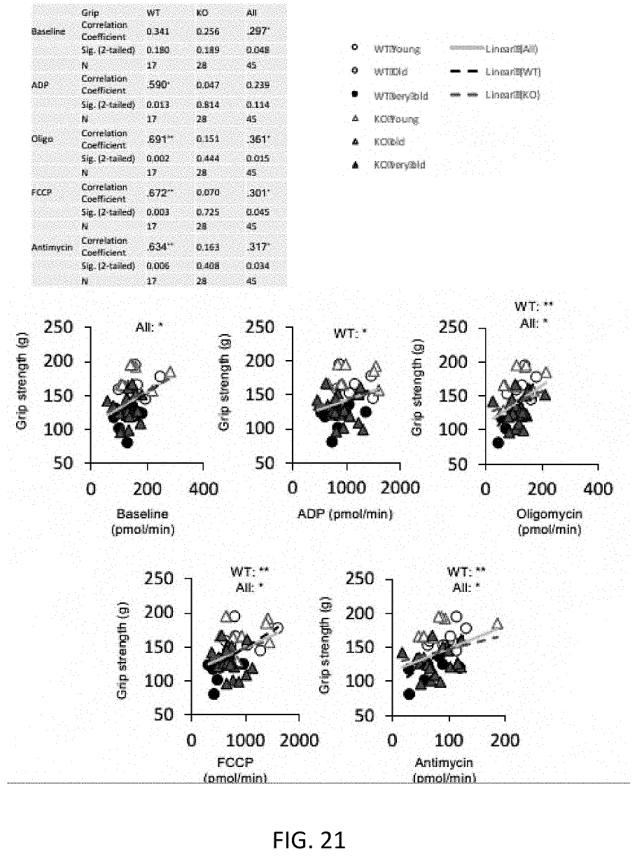

FIG. 21 shows grip strength (g) vs. oxygen consumption rate (OCR) were positively correlated in WT.

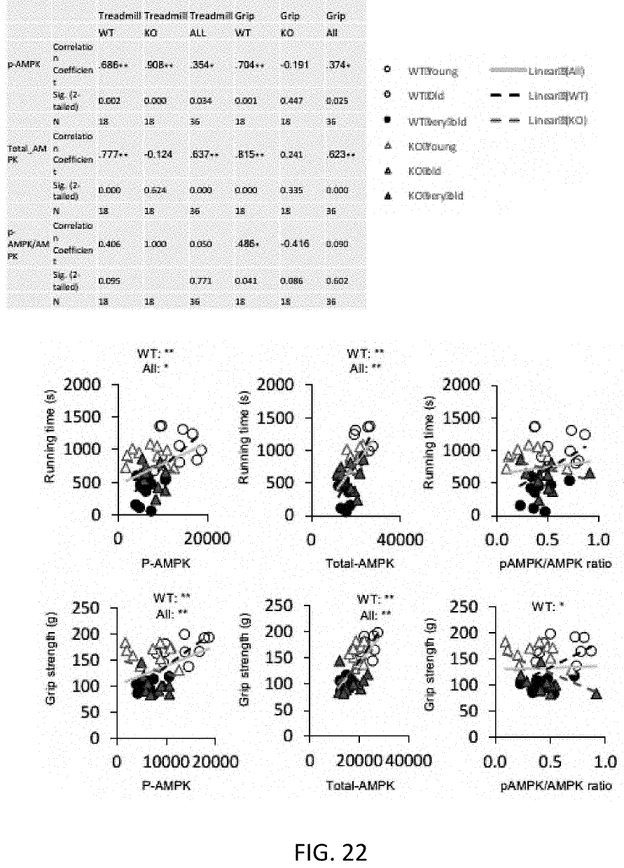

FIG. 22 shows treadmill running time (s) vs AMPK levels and grip strength (g) vs AMPK levels were positively correlated in WT mice (p-AMPK and AMPK).

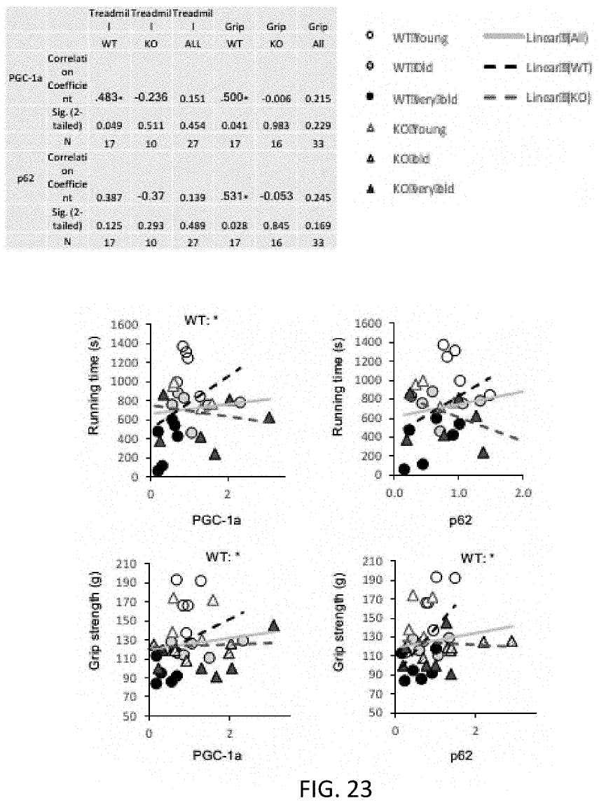

FIG. 23 shows treadmill running time (s) vs PGC-1a or p62 and grip strength (g) vs PGC-1a or p62 were positively correlated in WT mice (except for running time—p62).

In summary, age-related muscle dysfunction (strength and endurance) is attenuated by GHSR deletion. GHSR deletion also improves age-related decrease in fiber number (fast-twitched, 24m) and myogenin levels (30m). Mitochondrial dysfunction occurs with aging. GHSR deletion improves mitochondrial biogenesis, mitophagy and UPS marker (MuRF) in old mice. Age-related loss of NMJ is not prevented by GHSR deletion but GHSR knock out improves age-related changes in synaptic marker Argn. Young knockout mice show lower mitochondrial biogenesis, fusion and UPS than young WT mice in spite of preserved muscle function.

The loss of muscle mass and function is very common in the elderly, reducing overall functionality and quality of life, and increasing mortality. Old mice lacking a hormone receptor called GHSR-1a have better muscle function (can run longer on a treadmill) and have more muscle strength than regular old mice. This receptor can be targeted with drugs and, if these results are shown in humans, a new treatment for muscle function loss associated with old age and other conditions can be produced; thereby improving quality of life by allowing individuals to stay home longer, decreasing the need for hospitalizations and reducing the cost of healthcare. As there are no medications currently approved to improve muscle function, this discovery is of great clinical significance

3. Methods

i. Animals

Young (5-7-month-old), old (˜24-26m), and very old (˜28-30m) male growth hormone (GH) secretagogue receptor (GHSR)-1a wild type (Ghsr+/+) and knock out (Ghsr−/−) mice on a C57BL/6J background were used for the current study as previously described [1]. Mice were individually housed and maintained on a 12/12 light/dark cycle (lights on at 6 AM). A week before the experiments, mice were acclimated to their cages and human handling. Body weight, body composition, treadmill tests, and grip tests were evaluated during a week period before termination. A sub-cohort of mice was used for food intake measurements. Mice were euthanized by either a CO2-infused chamber or isoflurane followed by cervical dislocation. Hindlimb muscles were collected and weighed for estimating muscle mass and biochemical analysis. All experiments were conducted with the approval of the Institutional Animal Care and Use Committee at VA Puget Sound Health Care System and in compliance with the NIH Guidelines for Use and Care of Laboratory Animals.

ii. Food Intake, Body Weight and Body Composition

After a week of acclimation in their cages, the daily food consumption of each animal was evaluated for a week by weighing the food on top of the cages. Body weight and body composition were assessed by nuclear magnetic resonance (NMR, Bruker optics, The Woodlands, Tex.) one or two days before termination. Lean body mass and fat mass were calculated from a mean of two repeated measurements.

iii. Treadmill Test

The Exer-6 M treadmill was used for treadmill tests (Columbus Instruments). The protocol started with a 5 min-warm-up at a speed of 5 m/min. Following these 5 mins, speed was increased by 1 m/min every minute. The mouse was motivated to run by lightly tapping at the bottom using a cotton swab until exhausted. The total amount of time (sec) the mouse remained on the treadmill was identified.

iv. Grip Strength

Grip strength was measured right before euthanasia by a grip strength meter with a digital force gauge (Columbus Instruments, Columbus, Ohio). Forelimb grip strength was accessed by allowing the mouse to grasp a pull bar connected to a force gauge by only using its forelimbs. The maximum grip strength was recorded in the force gauge in kilograms. The test was performed three times at a one-minute interval. Maximum grip strength was recorded, and the final result is expressed as grip strength at endpoint normalized to baseline grip strength in %.

v. Immunohistochemistry

The cross-sectional area (CSA) of individual fibers from the plantaris (PL) muscle was determined as previously described [2-4]. Briefly, the OCT-mounted GAS/PL muscle was sliced at 10 μm using a Cryostat (Leica CM3050S, Nußloch, Germany) at −25° C. All muscles were transected at the mid-belly area, the largest cross-section of the whole muscle. The muscle sections were dehydrated for 30 minutes, and blocked with 10% goat serum in PBS for one hour. Primary antibodies were applied for two hours at room temperature. Primary antibodies and dilutions were used as follows: BA-F8 (1:50), which detects myosin heavy chain (MHC)-I fibers; SC-71 (1:600), which detects MHC-IIA fibers; and BF-F3 (1:100), which detects type MHC-IIB fibers (Developmental Studies Hybridoma Bank, Iowa City, Iowa). After three washes in PBS, sections were incubated in the corresponding secondary antibodies (Thermo Fisher Scientific, Waltham, Mass.) for 1 hour: Alexa 350 IgG2b (1:500) for MHC-I (blue); Alexa 488 IgG1 (1:500) for MHC-IIA (green); and Alexa 555 IgM (1:500) for MHC-IIB (red). After another three washes in PBS, the sections were mounted with Prolong Gold AntiFade reagent (Thermo Fisher Scientific).

The 10× plantaris muscle cross-sectional image was obtained by Nikon Ni-E microscope and NIS-Elements software (Nikon, Tokyo, Japan). To analyze the CSA of muscle fibers, approximately 100 type IIA, 200 type IIB, and 60-80 type IIX or IIA/X fibers from the whole PL area were analyzed using the same methods. The percentage of each fiber type in PL muscle was analyzed by using the Cell Counter plugin from the Image J analysis software (National Institutes of Health, http://rsb.info.nih.gov/ij/).

vi. Real-Time Reverse Transcription-Quantitative Polymerase Chain Reaction (RT-PCR)

Half of the GAS/PL muscle from the left side of the animal was saved in RNAlater® (Qiagen, Valencia, Calif.) after harvesting. RNA was isolated by using Qiagen RNeasy mini kit (Qiagen). Transcription levels of the isolated RNA were identified by BioTek Cytation 5. Total RNA was reverse transcribed to cDNA by QuantiTect Reverse Transcription Kit (Qiagen). RT-PCR was detected by an ABI 7500 instrument (Applied Biosystems, Foster City, Calif.) by using predesigned Taqman Expression Assays (Thermo Fisher Scientific, Waltham, Mass.). The quantification of genes of interest was normalized to a reference gene hypoxanthine guanine phosphoribosyl transferase (Hprt) and expressed as a relative fold-change of the Ghsr+/+ young group by a standard 2-ΔCT method. The following Taqman primers from Thermo Fisher Scientific (4331182) were used in this study: Myog, Myod1, Pax7, Ppargc1a, Opa1, Mfn2, Fis1, Dnm1l, Sqstm1, Bnip3, Fbxo32, Trim63, Becn1, Atg5, Atg7, Agm, Nog, Ncam1, and Musk.

vii. Western Blotting

Western blotting was performed to identify the protein content of LC3B, p62, Oxidative phosphorylation (OXPHOS), peroxisome proliferator-activated receptor-gamma coactivator (PGC-1α), total AMPK, and p-AMPK in GAS/PL muscles. A portion of GAS/PL muscle (˜60 mg) was homogenized in RIPA buffer (Thermo Fisher Scientific) with a cocktail of protease and phosphatase inhibitors (Thermo Fisher Scientific). The homogenate was centrifuged at 10,000 g for 15 minutes at 4° C., and the supernatant was collected as the protein extraction. For OXPHOS content, isolated mitochondria were used for Western blotting (see methods “Mitochondria isolation and mitochondrial respiration measurements”). The protein concentration was further quantified by a bicinchoninic acid (BCA) assay (Thermo Fisher Scientific) using bovine serum albumin (BSA) as a standard. Prior to electrophoresis, protein extractions were diluted with 5× Lane Marker Reducing Sample Buffer (Thermo Fisher Scientific) and heated at 95° C. for 4 minutes. 50 ug protein was loaded onto 4-15% Criterion™ TGX™ Precast Midi Protein Gels (BIO-RAD, Hercules, Calif.) and separated by using a BIO-RAD Criterion™ Cell electrophoresis system (165-6001). The proteins were then transferred to nitrocellulose membranes by a BIO-RAD Criterion™ Blotter (170-4070) at 100 V, 4° C., for 30 min. After blocking in 5% non-fat dry milk in TBS/T at room temperature for 1 hour, membranes were incubated with primary antibodies overnight at 4° C. The following primary antibodies and dilutions were used for the experiments: anti-LC3B antibody (1:500. NB100-2220, Novus Biologicals, CO); anti-SQSTM1/p62 antibody (1:1000. Ab56416, Abcam, Cambridge, Mass.); GAPDH (D16H11) XP® Rabbit mAb(horseradish peroxidase (HRP)-Conjugate, 1:2000. 8884, Cell Signaling, Beverly, Mass.); rabbit anti phospho AMPK (Thr 172, 1:1000, Cell Signaling, 2535); rabbit anti total AMPK (1:1000, Cell Signaling, 5831); Anti-PGC1 alpha antibody (1:1000, ab54481, Abcam); Total OXPHOS Rodent WB Antibody Cocktail (1:1000, ab110413, Abcam); Anti-beta Actin antibody (1:2000, ab8227, Abcam). Following incubation, the membranes were washed and then probed with the corresponding HRP-conjugated secondary antibodies (except GAPDH). The membranes were developed using SuperSignal™ West Dura Extended Duration Substrate (Thermo Fisher Scientific) and imaged by ImageQuant LAS 4000 (GE Health Care, Chicago, Ill.). The quantification of densitometry was analyzed by ImageJ and expressed as a ratio over GAPDH.

viii. Electrochemiluminescence Immunoassay

AMPK and p-AMPK (Thr172) in GAS muscles were detected by MULTI-ARRAY 96 Plate Pack, SECTOR Plate (Meso-Scale Diagnostics, L15XA-3, Rockville, Md.). The procedure was adapted from a previous publication by Esquejo et al [5]. In brief, each plate was prepared by overnight coating with 50 g anti-AMPK alpha 1+AMPK alpha 2 antibody [34.2] ab80039 at 4° C. for overnight. On the following day, the plate was washed with 1×MSD Tris wash buffer followed by incubating with 1% MSD Blocker A for 1 hour. After one more wash, 125 □g protein lysate was loaded and incubated at 37° C. for 2 hours. After three washes, 25 ng rabbit anti phospho AMPK (Thr 172, Cell Signaling, 2535) or 100 ng rabbit anti total AMPK (Cell Signaling, 5831) antibody was added and incubated for 1.5 hours at 37° C. After one wash, 50 ng MSD anti-rabbit SULFO-TAG antibody was added per well and incubated for 1 hour at 37° C. After another three washes, 1×MSD Read buffer was added and the plate was read on MSD Sector Imager (MSD).

ix. Mitochondria Isolation and Mitochondrial Respiration Measurements

The mitochondrial respiration tests were done in a separate cohort of mice as it requires plantaris muscles from both sides, and experiments for identifying molecular markers and CSA require a complete set of GAS/PL muscles. Therefore, CSA and molecular markers are from a set of mice separate from mice used for mitochondrial respiration. However, other data including BW, grip strength, muscle wet weight are combined data from both sets of mice as there was no difference in these outcomes between the two cohorts. The mitochondria isolation method is adapted from an established protocol published previously [6]. Plantaris muscles were harvested while the mouse was under deep anesthesia by isoflurane and immediately saved in mitochondria isolation buffer ((MIB), 210 mM sucrose, 2 mM EGTA, 40 mM NaCl, 30 mM HEPES, pH 7.4) on ice. The muscle was then homogenized in MIB by Kimble homogenizer and centrifuged at 900 g and 4° C. for 10 min. The supernatant was collected and centrifuged again at 10,000 g and 4° C. for 10 min. The mitochondrial pellet was collected and resuspended in MIB, and protein concentration was identified by Pierce Rapid Gold BCA Protein Assay (Thermo Fisher Scientific). After another centrifuge at 10,000 g and 4° C. for 10 min, isolated mitochondria were resuspended in mitochondrial assay solution ((MAS), 70 mM sucrose, 220 mM d-mannitol, 10 mM KH2PO4, 5 mM MgCl2, 2 mM HEPES, 1 mM EGTA, 0.2% fatty-acid free BSA, pH 7.4; Sigma-Aldrich, Carlsbad, Calif., USA) with substrates (5 mM malate and 5 mM pyruvate).

Mitochondrial respiration was detected by Agilent Seahorse XFe24 Extracellular Flux Assay Kit (Agilent Technologies, Santa Clara, Calif.) by using the methods adapted from Rodger's protocol [7, 8]. The day prior to the Seahorse experiment, XFe24 sensor cartridges were hydrated and incubated in a non-CO2 incubator overnight following the manufacturer's instructions. On the day of the experiment, the isolated mitochondria in MAS with substrates were plated in Agilent Seahorse XF24 Cell Culture Microplates (7.5 μg/well in triplicates). After centrifugation at 2,000 g and 4° C. for 20 min, an additional MAS with substrates were added to each well and made a final volume of 500 ul in each well. 50 ul of adenosine diphosphate (ADP), 55 ul oligomycin, 60 ul carbonyl cyanide-p-trifluoromethoxy-phenylhydrazone (FCCP), and 65 ul of antimycin A were loaded into the cartridge plate and injected into the cell plate in sequence with final concentrations as follows: ADP 4 mM, oligomycin 2 uM, FCCP 4 uM, and antimycin A 2 uM. Oxygen consumption rate ((OCR), pmol/min) was measured using an XFe24 Seahorse Instrument (Agilent Technologies) in real-time. Two measures were made in each mitochondrial respiration state: basal (state 2 respiration), phosphorylating respiration in the presence of ADP (state 3 respiration), resting respiration in the presence of oligomycin (state 4o respiration), maximal uncoupling respiration in the presence of FCCP (state 3u respiration) and electron transport chain-unrelated respiration in the presence of complex III inhibitor antimycin A.

x. Proteasome Activity

Quadricep (Quad) muscles were collected and used for proteasome activity assay. Protein extraction procedure was described in “Western blotting” except that protease inhibitor was omitted for this procedure. The chymotrypsin-like activity in Quad was detected by Proteasome Activity Assay Kit (Abcam, ab107921) by using an AMC-tagged peptide substrate (Succ-LLVY-AMC in DMSO). Samples with proteasome inhibitor MG-132 were served as a control. A protocol provided by the manufacturer was used for this assay. A serial dilution of 7-Amino-4-methylcoumarin (AMC, Sigma-Aldrich, St. Louis, Mo.) was used to generate a standard curve. Fluorescence was measured in 80 ug muscle lysate at 37° C. in Synergy™ HTX Multi-Mode Microplate Reader (BioTek, Winooski, Vt.) at a wavelength of 350/440 nm (Ex/Em) for 1 hour at 37° C. The activities were determined by comparing peptide fluorescence from samples with fluorescence of the standard curve of AMC.

xi. Quantification of Denervated Neuro-Muscular Junctions (NMJs) in EDL Muscles

After the mice were euthanized, EDL muscles were collected and blocked in 4% bovine serum albumin (Sigma) and 0.1% Triton X-100 (Roche) in PBS overnight at 4° C. while rotating. Then the muscles were incubated with the primary antibody detecting synaptic vesicle glycoprotein 2A (sv2, DSHB, 1:50) and neurofilament AB (1:50) overnight at 4° C. while rotating. After washing in PBS for 5 hours, muscles were incubated with the secondary antibody anti-Mouse IgG1, Alexa 568, (Thermo Fisher Scientific, A21124, 1:200) and α-Bungarotoxin (BTX), Alexa Fluor™ 488 conjugate (Thermo Fisher Scientific, 1:500) overnight at 4° C. while rotating (in dark). Muscles were then washed in PBS for overnight and mounted with Prolong Gold AntiFade reagent (Thermo Fisher Scientific). Confocal microscopy (Nikon A1R HD) was performed to identify innervated NMJs that had both red (synaptophysin) and green (BTX) staining, while denervated NMJs only had BTX stained. Images were taken at ×20 magnification and the whole detectable Z-disk of the sample (˜70-80 □m, 2 □m/step). Approximately 15 NMJs were analyzed per animal. Quantification of denervation was performed by ImageJ and the procedure was described previously by Tse et al [9].

xii. Statistics

Two-way analysis of variance (ANOVA) was performed to identify differences between genotypes (Ghsr+/+ vs. Ghsr−/−) across age groups (Young, Old and/or Very old) followed by Fisher's least significant difference (LSD) post hoc test (p<0.05). Spearman correlations were used to determine associations. Values are presented in mean±SE. All statistical testing was performed using IBM SPSS version 18 software.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Liu H, Luo J, Guillory B, Chen J A, Zang P, Yoeli J K, et al. Ghrelin ameliorates tumor-induced adipose tissue atrophy and inflammation via Ghrelin receptor-dependent and -independent pathways. Oncotarget. 2020; 11:3286-302. doi:10.18632/oncotarget.27705
2. Baumann C W, Liu H M, Thompson L V. Denervation-Induced Activation of the Ubiquitin-Proteasome System Reduces Skeletal Muscle Quantity Not Quality. PLoS One. 2016; 11:e0160839. doi:10.1371/journal.pone.0160839
3. Liu H M, Ferrington D A, Baumann C W, Thompson L V. Denervation-Induced Activation of the Standard Proteasome and Immunoproteasome. PLoS One. 2016; 11:e0166831. doi:10.1371/journal.pone.0166831
4. Sawano S, Komiya Y, Ichitsubo R, Ohkawa Y, Nakamura M, Tatsumi R, et al. A One-Step Immunostaining Method to Visualize Rodent Muscle Fiber Type within a Single Specimen. PLoS One. 2016; 11:e0166080. doi:10.1371/journal.pone.0166080
5. Esquejo R M, Salatto C T, Delmore J, Albuquerque B, Reyes A, Shi Y, et al. Activation of Liver AMPK with PF-06409577 Corrects NAFLD and Lowers Cholesterol in Rodent and Primate Preclinical Models. EBioMedicine. 2018; 31:122-32. doi:10.1016/j.ebiom.2018.04.009
6. Chavez J D, Tang X, Campbell M D, Reyes G, Kramer P A, Stuppard R, et al. Mitochondrial protein interaction landscape of SS-31. Proc Natl Acad Sci USA. 2020; 117:15363-73. doi:10.1073/pnas.2002250117
7. Rogers G W, Brand M D, Petrosyan S, Ashok D, Elorza A A, Ferrick D A, et al. High throughput microplate respiratory measurements using minimal quantities of isolated mitochondria. PLoS One. 2011; 6:e21746. doi:10.1371/journal.pone.0021746
8. Boutagy N E, Rogers G W, Pyne E S, Ali M M, Hulver M W, Frisard M I. Using Isolated Mitochondria from Minimal Quantities of Mouse Skeletal Muscle for High throughput Microplate Respiratory Measurements. J Vis Exp. 2015; e53216. doi:10.3791/53216
9. Tse N, Morsch M, Ghazanfari N, Cole L, Visvanathan A, Leamey C, et al. The neuromuscular junction: measuring synapse size, fragmentation and changes in synaptic protein density using confocal fluorescence microscopy. J Vis Exp. 2014; doi:10.3791/52220

1. A method of treating muscle loss in a subject comprising administering a therapeutically effective amount of a GHSR1a antagonist, a GHSR1a inverse agonist, or a decoy ghrelin receptor to a subject in need thereof.

2. The method of claim 1, wherein muscle loss is associated with sarcopenia or cachexia.

3. The method of claim 1, wherein the subject in need thereof is 65 or older.

4. The method of claim 1, wherein the subject in need thereof has or has been diagnosed with a tumor or cancer or other condition associated to cachexia including but not limited to heart failure, liver failure, kidney failure, dementia, sepsis or other acute or chronic conditions.

5. The method of claim 1, wherein the subject in need thereof has a neurological condition.

6. The method of claim 1, wherein the GHSR1a antagonist is a protein, nucleic acid, fatty acid, or chemical compound that binds GHSR1a and/or prevents GHSR1a activation.

7. The method of claim 1, wherein the GHSR1a antagonist is a LEAP-2 related compound, (d-Lys-3)-GHRP-6, JMV 2959, HM-04, YIL 781 hydrochloride among others.

8. The method of claim 1, wherein the GHSR1a inverse agonist is a protein, nucleic acid, fatty acid, or chemical compound that binds to GHSR1a and prevents or opposes ghrelin's effects on GHSR1a.

9. The method of claim 1, wherein the GHSR1a inverse agonist is PF 04628935, PF 05190457, MSP, LEAP2 (liver-expressed antimicrobial peptide 2).

10. The method of claim 1, wherein the decoy ghrelin receptor is a receptor that binds ghrelin and does not activate GHSR1a.

11. The method of claim 1, wherein the decoy ghrelin receptor is a GHSR1a fusion protein.

12. The method of claim 1, wherein the administration of the therapeutically effective amount of a GHSR1a antagonist, GHSR1a inverse agonist or decoy ghrelin receptor increases a muscle function marker in the subject in need thereof.

13. The method of claim 12, wherein the muscle function marker is myogenin or PGC-1α.

14. A method of inhibiting the binding of ghrelin to GHSR1a in a subject comprising administering a therapeutically effective amount of GHSR1a antagonist, a GHSR1a inverse agonist or a decoy ghrelin receptor to a subject in need thereof.

15. The method of claim 14, wherein the GHSR1a antagonist is a protein, nucleic acid, fatty acid, or compound that binds GHSR1a and prevents GHSR1a activation.

16. The method of claim 14, wherein the GHSR1a antagonist is a LEAP-2 related compound, (d-Lys-3)-GHRP-6, JMV 2959, HM-04, or YIL 781 hydrochloride.

17. The method of claim 14, wherein a GHSR1a inverse agonist is a protein, nucleic acid or compound that binds to GHSR1a and prevents or opposes ghrelin's effects on GHSR1a.

18. The method of claim 17, wherein a GHSR1a inverse agonist is PF 04628935, PF 05190457, MSP, or LEAP2 (liver-expressed antimicrobial peptide 2).

19. The method of claim 14, wherein a decoy ghrelin receptor is a receptor that binds ghrelin and does not activate GHSR1a.

20. The method of claim 19, wherein a decoy ghrelin receptor is a GHSR1a fusion protein.

21.-41. (canceled)
